(19) United States
(12) Patent Application Publication
(10) Pub. No.: US 2026/0132474 A1
(43) Pub. Date: May 14, 2026

(54) MOLECULAR MARKER RELATED TO IMMUNE TRAITS USED AS MARKER-ASSISTED SELECTION FOR YAKS AND USE THEREOF

(71) Applicant: LANZHOU Institute of Husbandry and Pharmaceutical Sciences of CAAS, Lanzhou (CN)

(72) Inventors: Chunnian LIANG, Lanzhou (CN); Daoning YU, Lanzhou (CN); Xue ZHAO, Lanzhou (CN); Yan YANG, Lanzhou (CN); Xiaoming MA, Lanzhou (CN); Yongfu LA, Lanzhou (CN); Xiaoyun WU, Lanzhou (CN); Min CHU, Lanzhou (CN); Xita ZHA, Lanzhou (CN); Yuansheng ZHAO, Lanzhou (CN); Xuemei LIU, Lanzhou (CN)

(73) Assignee: LANZHOU Institute of Husbandry and Pharmaceutical Sciences of CAAS, Lanzhou (CN)

(21) Appl. No.: 19/344,634

(22) Filed: Sep. 30, 2025

(30) Foreign Application Priority Data
Nov. 8, 2024 (CN) 202411588368.4

(51) Int. Cl. C12Q 1/6888; C12Q 1/6806; C12Q 1/6827; C12Q 1/686; C12Q 1/6869

(52) U.S. Cl. CPC C12Q 1/6888; C12Q 1/6806; C12Q 1/6827; C12Q 1/686; C12Q 1/6869; C12Q 2600/124; C12Q 2600/156

(57) ABSTRACT

A molecular marker related to immune traits used as marker-assisted selection for yaks and a use thereof are provided. A SNP locus related to immune traits in yaks is located at base 163,688,148 on chromosome 1 of the yak genome assembly LU_Bosgru_v3.0. The method for detecting immune traits in yaks using a SNP locus and a molecular marker based on the locus has the advantages of high accuracy, fast detection speed, low cost, and easy result judgment compared with the traditional detection method. A new SNP molecular marker resource is provided for marker-assisted selection for immune traits in yaks for non-diagnostic purposes.

### CROSS REFERENCE TO THE RELATED APPLICATIONS

This application is based upon and claims priority to Chinese Patent Application No. 202411588368.4, filed on Nov. 8, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC315_SequenceListing.xml, created on Sep. 19, 2025, and is 4,316 bytes in size.

### TECHNICAL FIELD

The present invention belongs to the technical field of molecular biology detection, and in particular, to a molecular marker related to immune traits used as marker-assisted selection for yaks and use thereof.

### BACKGROUND

Yak is one of the important livestock breeds in the alpine pastoral areas of the Qinghai-Tibet Plateau and an important part of plateau animal husbandry. Yaks have adapted to extreme natural environments, such as high altitude, low air pressure, strong ultraviolet rays, lack of oxygen and malnutrition. Therefore, the yaks have strong survival abilities and provide pastoral areas with a variety of production and living materials such as meat, milk, wool and leather. Yaks are not only the guarantee of life for plateau herdsmen, but also an important symbol of their wealth. Meanwhile, yaks are a precious gene treasure trove genetically, with unique biological characteristics, strong physique and high adaptability to harsh environments.

At present, the yak industry still has problems with insufficient selection and breeding in terms of production traits such as disease resistance and fertility. The yak population is small, which makes it difficult to meet market demand. Meanwhile, the construction of breeding technology system is relatively backward, which restricts the sustainable development of the yak industry. These problems have become one of the key factors restricting the development of the yak industry.

Therefore, there is an urgent need to develop a modern breeding method for yaks, especially a breeding strategy targeting disease resistance of yaks. Genes related to disease resistance are screened, and molecular genetics are used for marker-assisted selection, which accelerates the genetic improvement of yaks, promote the breeding of new breeds and the healthy development of the yak industry.

Basic genetic research on disease resistance, combined with molecular biology and genetic engineering technology, can effectively enhance the resistance and immunity of yaks to pathogens, thereby reducing the threat of diseases to yaks' health, reducing economic losses, and promoting the development of the yak industry in an efficient and sustainable direction.

### SUMMARY

An objective of the present invention is to provide a molecular marker related to immune traits used as marker-assisted selection for yaks and use thereof.

To achieve the above objective, the present invention provides the following technical solutions.

The present invention provides a single nucleotide polymorphism (SNP) molecular marker related to immune traits in yaks, the SNP molecular marker is located at base 163,688,148 on chromosome 1 of the yak genome assembly LU_Bosgru_v3.0, and a mutation base is C or T.

Preferably, when a base of the SNP locus is C, a genotype is CC or CT; when a base of the SNP locus is T, the genotype is TT; when the genotype is TT, immunoglobulin A (IgA) and immunoglobulin G (IgG) of a yak individual are significantly higher than those of the genotype CCl and when the genotype is TT, IgG of a yak individual is significantly higher than that of the genotype CT.

The present invention further provides use of the SNP molecular marker in preparation of a product for detecting yak immunity or a product for assisting yak breeding.

The present invention further provides a primer pair for amplifying the SNP molecular marker, wherein a sequence of the primer pair is shown as SEQ ID NOS: 2-3.

The present invention further provides use of the primer pair in preparation of a product for detecting yak immunity or a product for assisting yak breeding.

The present invention further provides a kit for detecting immunity of yaks, which includes a reagent for detecting the SNP molecular marker or the primer pair.

The present invention further provides a kit for assisting yak breeding, which includes a reagent for detecting the SNP molecular marker or the primer pair.

The present invention further provides a method for marker-assisted selection of immune traits in yaks for non-diagnostic purposes, which includes the following steps:
(1) extracting genomic deoxyribonucleic acid (DNA) of yaks;
(2) performing amplification by taking the genomic DNA of the yaks obtained in the step (1) as a template and using the primer pair to obtain an amplified product; and
(3) performing genotype analysis on the amplified product to obtain yaks with different genotypes, and correlating the genotypes of the yaks with immune indicators; wherein immunoglobulin is IgA or IgG.

Preferably, the amplification system in the step (2) is 25 μL in total, including: 12.5 μL of 2×L-Exp Taq Master Mix, 8.5 μL of RNase free water, 1 μL of an upstream primer, 1 μL of a downstream primer, and 2 μL of the template.

Preferably, the amplification program in the step (2) is as follows: 94° C. for 1 min; 98° C. for 10 s, 58° C. for 30 s, 72° C. for 1 min, for a total of 35 cycles; and extension at 72° C. for 2 min.

Compared with the prior art, the present invention has the following beneficial effects.

The present invention provides a molecular marker related to immune traits used as marker-assisted selection for yaks and use thereof. According to the technical solution of the present invention, the method for detecting immune traits in yaks using a SNP locus and a molecular marker based on the locus has the advantages of high accuracy, fast detection speed, low cost, and easy result judgment compared with the traditional detection method, which provides a new SNP molecular marker resource for marker-assisted selection for immune traits in yaks for non-diagnostic purposes.

### BRIEF DESCRIPTION OF THE DRAWINGS

To more clearly illustrate the technical solutions in the embodiments of the present invention or in the prior art, the drawings required to be used in the description of the embodiments or the prior art are briefly introduced below. It is obvious that the drawings in the description below are merely embodiments of the present invention, and those of ordinary skill in the art can obtain other drawings according to the drawings provided without creative efforts.

FIG. 1 shows a PCR amplified product: M represents Marker; 1, 2, and 3 represent three groups of repeats.

FIG. 2 is a peak diagram and a sequence obtained after sequencing of the PCR product, where AGGCGGA is shown as SEQ ID NO: 4, and AGGTGGA is shown as SEQ ID NO: 5.

### DETAILED DESCRIPTION OF THE EMBODIMENTS

The present invention provides a SNP molecular marker related to immune traits in yaks, the SNP molecular marker is located at base 163,688,148 on chromosome 1 of the yak genome assembly LU_Bosgru_v3.0, and a mutation base is C or T.

In the present invention, when abase of the SNP locus is C, a genotype is CC or CT; when a base of the SNP locus is T, the genotype is TT; when the genotype is TT, IgA and IgG of a yak individual are significantly higher than those of the genotype CCl and when the genotype is TT, IgG of a yak individual is significantly higher than that of the genotype CT.

The present invention further provides use of the SNP molecular marker in preparation of a product for detecting yak immunity or a product for assisting yak breeding.

The present invention further provides a primer pair for amplifying the SNP molecular marker, wherein a sequence of the primer pair is shown as SEQ ID NOS: 2-3.

The present invention further provides use of the primer pair in preparation of a product for detecting yak immunity or a product for assisting yak breeding.

The present invention further provides a kit for detecting immunity of yaks, which includes a reagent for detecting the SNP molecular marker or the primer pair.

The present invention further provides a kit for assisting yak breeding, which includes a reagent for detecting the SNP molecular marker or the primer pair.

The present invention further provides a method for marker-assisted selection of immune traits in yaks for non-diagnostic purposes, which includes the following steps:
(1) extracting genomic DNA of yaks;
(2) performing amplification by taking the genomic DNA of the yaks obtained in the step (1) as a template and using the primer pair to obtain an amplified product; and
(3) performing genotype analysis on the amplified product to obtain yaks with different genotypes, and correlating the genotypes of the yaks with immune indicators; wherein immunoglobulin is IgA or IgG.

In the present invention, the amplification system in the step (2) is 25 μL in total, including: 12.5 μL of 2× L-Exp Taq Master Mix, 8.5 μL of RNase free water, 1 μL of an upstream primer, 1 L of a downstream primer, and 2 μL of the template.

In the present invention, the amplification program in the step (2) is as follows: 94° C. for 1 min; 98° C. for 10 s, 58° C. for 30 s, 72° C. for 1 min, for a total of 35 cycles; and extension at 72° C. for 2 min.

The technical solutions provided by the present invention will be described in detail below with reference to examples, which, however, should not be construed as limiting the scope of the present invention.

### Example 1

### 1. Sample Collection

The Niangya yak was selected as a test object. 5 mL of blood samples from 192 fasting yaks were collected from a ranch in Jiali County, Nagqu City, Tibet Autonomous Region, and placed in a clean procoagulant vacuum blood collection tube. The tube was left to stand for 30 min and then centrifuged at 3500 r/min for 10 min. The supernatant was aspirated into a PE tube, sealed and stored in a −20° C. low-temperature refrigerator; another 5 mL of blood sample was collected in a blood collection tube with EDTA-K2 anticoagulant added. After the collection was completed, the blood samples were quickly mixed, placed in a sampling box containing an ice pack for temporary storage, and transported back to a laboratory and stored in a −20° C. refrigerator for genomic DNA extraction.

### 2. Main Reagents and Instruments

EDTA-K2 vacuum blood collection tubes were purchased from Jiangsu Yuli Medical Equipment Co., Ltd.; blood genomic DNA extraction kit was purchased from Tiangen Biotech (Beijing) Co., Ltd.; NanoDrop 2000 spectrophotometer was purchased from Thermo Fisher Scientific, USA; DL2000 Marker, agarose, and nucleic acid dye were purchased from Beijing Solarbio Science & Technology Co., Ltd.; 2× L-Exp Taq Master Mix (dye plus) was purchased from Hunan Aikori Biotechnology Co., Ltd.; electrophoresis instrument was purchased from Beijing Liuyi Instrument Factory; and PCR instrument was purchased from BioRad. IgA (MB-4907A), IgG (MB-4616A), and immunoglobulin M (IgM) (MB-4908A) detection kits were purchased from Jiangsu Meibiao Biotechnology Co., Ltd.

### 3. Method

3.1. Detection of immunoglobulins IgA, IgG, and IgM

The determination was performed using a double-antibody one-step sandwich method according to the IgA, IgG, and IgM detection kits from Jiangsu Meibiao Biotechnology Co., Ltd. First, the required strips were taken out from an aluminum foil bag after equilibration at room temperature for 20 min, and the remaining strips were sealed in a ziplock bag and placed at 4° C. Standard wells and sample wells were set up, and 50 μL of standard with different concentrations was added to each standard well; 10 μL of the sample to be tested was first added to the sample well, and then 40 μL of the sample diluent was added; nothing was added to blank wells. Except for the blank wells, 100 μL of horseradish peroxidase (HRP)-labeled detection antibody was added to each of the standard wells and sample wells, and the reaction wells were sealed with a sealing film and incubated in a 37° C. water bath or incubator for 60 min. The liquid was discarded, and the plate was blotted dry on absorbent paper. Each well was then completely filled with washing buffer, incubated for 1 min, after which the buffer was decanted and the plate was blotted dry again. This washing procedure was repeated for 5 cycles (alternatively, an automated plate washer could be used). 50 μL of substrate A and B were added to each well and incubated at 37° C. in the dark for 15 min. 50 μL of stop solution was added to each well, and the OD value of each well was measured at a wavelength of 450 nm within 15 min. Finally, a standard curve was drawn. In the Excel worksheet, the standard concentration was used as the horizontal axis and the corresponding OD value was used as the vertical axis to draw the standard linear regression curve, and the IgA, IgG, and IgM concentration values of each sample were calculated according to the curve equation.

3.2. Extraction of Genomic DNA from Blood

The blood genomic DNA extraction kit from Tiangen Biotech (Beijing) Co., Ltd. was used to extract genomic DNA from the blood samples. The extracted DNA was placed under an ultraviolet spectrophotometer to detect the concentration and purity. The concentration >20 ng/L and OD₂₆₀/OD₂₈₀ between 1.7 and 1.9 met the experimental requirements. The extracted DNA was stored at −20° C. for future use.

### 3.3. Primer Design

A pair of specific primers was designed with genomic DNA as template using primer premier5.0 software.

### Sequences of Specific Primers:

(SEQ ID NO: 2)
5′-ACTAAGATCCCATGAGCCA-3′
(SEQ ID NO: 3)
5′-ATTGAATGAGACACCACCAG-3′

A length of the amplified fragment was 519 bp, and the primers were synthesized by Beijing Tsingke Biotechnology Co., Ltd.

### 3.4. PCR Amplification and Sequencing

The PCR amplification system was 25 μL in total, including: 12.5 μL of 2× L-Exp Taq Master Mix(dye plus), 8.5 μL of RNase free water, 1 μL of an upstream primer, 1 μL of a downstream primer, and 2 μL of a template.

The amplification program was as follows: 94° C. for 1 min; 98° C. for 10 s, 58° C. for 30 s, 72° C. for 1 min, for a total of 35 cycles; and extension at 72° C. for 2 min.

The PCR products were detected by 1% agarose gel electrophoresis. After the PCR products were qualified by the agarose gel electrophoresis, the sequencing was performed by direct sequencing, which was performed by Beijing Tsingke Biotechnology Co., Ltd.

The sequencing results of PCR products were compared using the biological analysis software MEGA 11.0 to analyze the sequencing peaks and complete the typing.

### 4. Statistical Analysis

According to the genotyping results, the number of individuals with different genotypes at each site was counted. The g163688148C>T gene frequency, genotype frequency, effective number of alleles (Ne), locus heterozygosity (He), and Hardy-Weinberg equilibrium test were calculated using Popgen32 software, and polymorphism information content (PIC) was calculated using software. The correlations between different genotypes of yaks and immunoglobulins IgA, IgG, and IgM were analyzed using the general linear model in IBM SPSS Statistics 26 software, and the results were expressed as “mean±standard error”.

### 5 Results

### 5.1. PCR Amplification and Sequencing Results

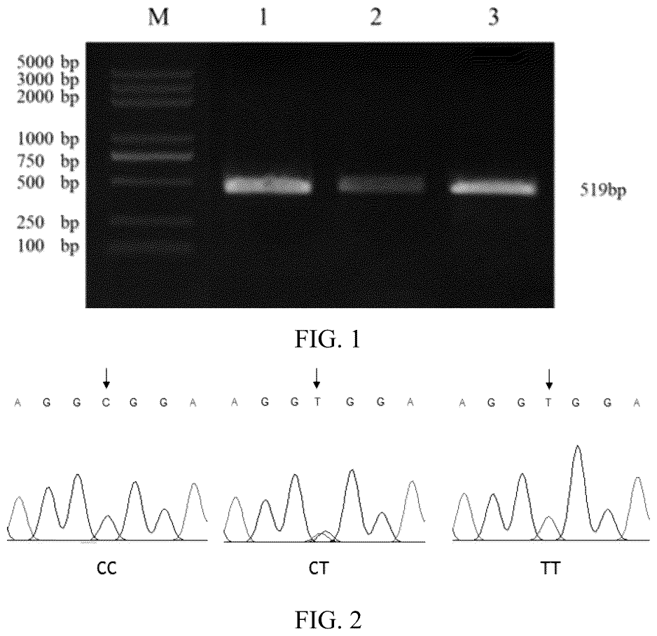

The amplified product of the g163688148C>T site on chromosome 1 of yak was detected using 1% agarose gel (see FIG. 1). The bands were clear without any mixed bands, with good specificity. The length of PCR product fragment was 519 bp, which was in line with the expected size, and the next step of the experiment could be performed.

The sequence of SEQ ID NO: 1 is:

ACTAAGATCCCATGAGCCACAGAGGAACAACTAAGCCCGTGCACTGCAG
CTAGAGAGTCTGAGGGCAGCGAGGAAGGTCCCGCATGCTGAAACTAAGA
CCCGACGCAGCCCAATAAATAAGCAAAATGCTGTGGTGATGCTACACGG
TGACCTCGTCCCCCCACCTAGCTTTCTGTAAAGCCCCACACTCAGGCGG
AACCACTGCTGCGGACAGGCAACAGGCGGGCAGGTGATCTTGTCTTGTG
AAAATCAATTCAGGGCTCCTAGAGCTCCTGCCGTGTGTGGCCCATTGGA
GTGGCCCTGCAGCGCCTCGGCCAGCCAGCTCCTAGGTCCTCACGGCCAT
CAGGAGGGTGGCCTGGGGCTTCGATGTCCTCTGTCCACACCCTCTCCCG
GACGGTCTCACTCACTCATGTGGTTCCACGTGCTGTGGGCTTTCACACC
TCCAGCTGGCCCTCCAGCTGATCTGACCCAGGCATTCACCTGCCCACTG
GGCATCTTCCTGGTGGTGTCTCATTCAAT

The peak diagram and sequence obtained after the PCR product was purified and sequenced are shown in FIG. 2. As shown in FIG. 2, a C-T mutation occurred at the g163688148C>T site, and there were three genotypes: CC, CT, and TT.

### 5.2. Statistical Analysis Results

The genotype and allele frequencies of the g163688148C>T SNP locus on chromosome 1 of yak were analyzed from the perspective of population genetics. It can be seen from Table 1 that, at the g163688148C>T SNP locus, the CC genotype frequency was the highest, which was the dominant genotype, and the C allele frequency was 65.9%, which was the dominant allele. The χ² fitness test showed that the SNP locus was in Hardy-Weinberg equilibrium (P>0.05) (Table 1). The expected heterozygosity of this locus was 0.449, the PIC was 0.348, and 0.25<PIC<0.50, which was moderately polymorphic.

TABLE 1
Polymorphism of g163688148C > T SNP locus on chromosome 1 of yak

| | | | | Allele | | | | | | |
|---|---|---|---|---|---|---|---|---|---|---|
| | Genotype frequency | | | frequency | | | | | | |
| Locus | CC | CT | TT | C | T | χ² | P | PIC | He | Ne |
| g163688148C > T | 0.448 | 0.422 | 0.130 | 0.659 | 0.341 | 0.727 | 0.394 | 0.348 | 0.449 | 1.186 |
| | (86) | (81) | (25) | | | | | | | |

### 5.3 Correlation Analysis Between Different Genotypes and Immunoglobulins IgA, IgG, and IgM

The correlation between different genotypes of yaks and the content of immunoglobulins IgA, IgG, and IgM was analyzed using the general linear model in IBM SPSS Statistics26 software. The results showed that, the IgA and IgG of yak individuals with genotype TT were significantly higher than those with genotype CC (p<0.05), and the IgG of yak individuals with genotype TT was significantly higher than that of genotype CT (p<0.05), while the IgA and IgG between CC and CT genotype yaks did not show significant differences (p>0.05), and the IgM of yak individuals with three genotypes had no significant differences (p>0.05), which indicated that the base of the g163688148C>T site on chromosome 1 of yak was significantly correlated with IgA and IgG of yak (p<0.05), and was a SNP marker related to yak. The results are shown in Table 2.

TABLE 2
Correlation analysis between different
genotypes and IgA, IgG, IgM

Genotypes

| Traits | CC | CT | TT |
|---|---|---|---|
| IgA (μg/mL) | 1119.21 ± | 1169.21 ± | 1233.76 ± |
| | 19.01b | 22.05ab | 42.62a |
| IgG (mg/mL) | 2.28 ± | 2.35 ± | 2.78 ± |
| | 0.07b | 0.06b | 0.16a |
| IgM (μg/mL) | 2069.59 ± | 2129.82 ± | 2203.38 ± |
| | 32.82 | 30.65 | 52.87 |

Note: Different lowercase letters between data in the same row indicate significant differences (P<0.05).

The SNP molecular marker described in the present invention is located at base 163,688,148 on chromosome 1 of the yak genome assembly LU_Bosgru_v3.0, a variation type is C/T, named g163688148C>T, and there are three genotypes. When the base 163688148 on chromosome 1 is C, the genotype is CC or CT; when the base 163688148 on chromosome 1 is T, the genotype is TT. Through the correlation analysis between different genotypes and IgA, IgG, and IgM contents, it is found that the IgA and IgG of yak individuals with genotype TT are significantly higher than those with genotype CC (p<0.05), and the IgG of yak individuals with genotype TT is significantly higher than that with genotype CT (p<0.05), while there is no significant difference in IgA and IgG between CC and CT genotype yaks (p>0.05), and there was no significant difference in IgM among yaks with the three genotype (p>0.05). The detection of the base at nucleotide site163688148 on chromosome 3 of the yak may determine the contents of IgA and IgG of the yak individual. The present invention provides a new SNP molecular marker resource for marker-assisted selection for immune traits in yaks for non-diagnostic purposes.

According to the above example, the present invention provides a molecular marker related to immune traits used as marker-assisted selection for yaks and use thereof. According to the technical solution of the present invention, the method for detecting immune traits in yaks using a SNP locus and a molecular marker based on the locus has the advantages of high accuracy, fast detection speed, low cost, and easy result judgment compared with the traditional detection method, which provides a new SNP molecular marker resource for marker-assisted selection for immune traits in yaks for non-diagnostic purposes.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A use of a reagent for detecting a single nucleotide polymorphism (SNP) molecular marker in preparation of an in-vitro detection reagent for immune traits of Niangya yaks or an assisted breeding detection reagent for the immune traits of the Niangya yaks, wherein the SNP molecular marker is located at base 163,688,148 on a first chromosome of Niangya yak reference genome assembly LU_Bosgru_v3.0, and a mutant base is C or T; and when a genotype is TT, immunoglobulin A (IgA) and immunoglobulin G (IgG) of a yak individual with the genotype TT are significantly higher than IgA and IgG of a yak individual with a genotype CC, and when the genotype is TT, the IgG of the yak individual with the genotype TT is significantly higher than IgG of a yak individual with a genotype CT.

2. The use according to claim 1, wherein the primer pair for detecting the SNP molecular marker is shown as SEQ ID NOS: 2-3.

3. A method for marker-assisted selection of immune traits in Niangya yaks for non-diagnostic purposes, comprising the following steps:
(1) extracting genomic deoxyribonucleic acid (DNA) of the Niangya yaks;
(2) performing amplification by taking the genomic DNA of the Niangya yaks obtained in the step (1) as a template and using the primer pair shown in SEQ ID NOS: 2-3 to obtain an amplified product; and
(3) performing genotype analysis on the amplified product to obtain the Niangya yaks with different genotypes, and correlating the different genotypes of the Niangya yaks with immune indicators; wherein an immunoglobulin is IgA or IgG;
wherein a SNP molecular marker is located at base 163,688,148 on a first chromosome of Niangya yak reference genome assembly LU_Bosgru_v3.0, and a mutant base is C or T; and when a genotype is TT, IgA and IgG of a yak individual with the genotype TT are significantly higher than IgA and IgG of a yak individual with a genotype CC, and when the genotype is TT, the IgG of the yak individual with the genotype TT is significantly higher than IgG of a yak individual with a genotype CT.

4. The method according to claim 3, wherein an amplification system in the step (2) is 25 μL in total, comprising: 12.5 μL of 2× L-Exp Taq Master Mix, 8.5 μL of RNase free water, 1 μL of an upstream primer, 1 μL of a downstream primer, and 2 μL of the template.

5. The method according to claim 3, wherein an amplification program in the step (2) is as follows: 94° C. for 1 min; 98° C. for 10 s, 58° C. for 30 s, and 72° C. for 1 min, for a total of 35 cycles; and extension at 72° C. for 2 min.


SEQUENCE LISTING (3 sequences)

SEQ ID NO: 1 - DNA, 519 bp, synthetic construct
  source 1..519: mol_type = other DNA
actaagatcccatgagccacagaggaacaactaagcccgtgcactgcagctagagagtctgagggcagcgaggaaggtcccgcatgctgaaactaagacccgacgcagcccaataaataagcaaaatgctgtggtgatgctacacggtgacctcgtccccccacctagctttctgtaaagccccacactcaggcggaaccactgctgcggacaggcaacaggcgggcaggtgatcttgtcttgtgaaaatcaattcagggctcctagagctcctgccgtgtgtggcccattggagtggccctgcagcgcctcggccagccagctcctaggtcctcacggccatcaggagggtggcctggggcttcgatgtcctctgtccacaccctctcccggacggtctcactcactcatgtggttccacgtgctgtgggctttcacacctccagctggccctccagctgatctgacccaggcattcacctgcccactgggcatcttcctggtggtgtctcattcaat

SEQ ID NO: 2 - DNA, 19 bp, synthetic construct
  source 1..19: mol_type = other DNA
actaagatcccatgagcca

SEQ ID NO: 3 - DNA, 20 bp, synthetic construct
  source 1..20: mol_type = other DNA
attgaatgagacaccaccag